(19) United States
(12) Patent Application Publication
(10) Pub. No.: US 2018/0071273 A1
(43) Pub. Date: Mar. 15, 2018

(54) NUTRITIONAL COMPOSITIONS TO ENHANCE MITOCHONDRIAL ENERGY PRODUCTION

(71) Applicant: SPECCIALTY NUTRITION GROUP, INC., Lighthouse Point, FL (US)

(72) Inventor: Gregory T. Horn, Lighthouse Point, FL (US)

(21) Appl. No.: 15/558,928

(22) PCT Filed: Mar. 15, 2016
(86) PCT No.: PCT/US16/22484

§ 371 Date: Sep. 15, 2017

Related U.S. Application Data
(60) Provisional application No. 62/134,449, filed on Mar. 17, 2015.

(51) Int. Cl. A61K 31/455; A23L 33/105; A23L 33/15; A61K 31/09; A61K 31/14; A61K 31/385; A61K 31/706; A61K 36/068; A61K 36/258; A61K 36/28; A61K 36/38; A61K 36/53; A61K 36/54; A61K 36/87; A61K 36/9066

(52) U.S. Cl. CPC A61K 31/455; A23V 2250/7046; A23L 33/15; A61K 31/09; A61K 31/14; A61K 31/385; A61K 31/706; A61K 36/068; A61K 36/258; A61K 36/28; A61K 36/38; A61K 36/53; A61K 36/54; A61K 36/87; A61K 36/9066; A61K 2300/00; A23V 2002/00; A23V 2250/21; A23V 2200/308; A23V 2250/0612; A23L 33/105

(57) ABSTRACT

The present disclosure provides nutritional compositions comprising a synergistic effective amount of a NAD− pre-cursor in a combination with an ATP booster. ‘The disclosure provides methods and formulations to enhance natural energy. The disclosure also provides methods and formulations treat mitochondrial energy disorders or diseases.

### CROSS REFERENCE TO RELATED APPLICATIONS

This application claims the benefit of U.S. Provisional Patent Application Ser. No. 62/134,449, filed Mar. 17, 2015, and entitled NUTRITIONAL COMPOSITIONS TO ENHANCE MITOCHONDRIAL ENERGY, which is incorporated herein by reference in its entirety.

### FIELD OF THE TECHNOLOGY

The present application generally relates to nutritional compositions to improve mitochondrial energy production and efficiency.

### BACKGROUND

Mitochondrial disease is a group of disorders caused by dysfunctional mitochondria, the organelles that generate energy for the cell. Mitochondria are found in every cell of the human body except red blood cells, and convert the energy of food molecules into the ATP that powers most cell functions. Mitochondrial diseases take on unique characteristics both because of the way the diseases are often inherited and because mitochondria are so critical to cell function. Some common symptoms are poor growth, loss of muscle coordination, muscle weakness, visual problems, hearing problems, learning disabilities, heart disease, liver disease, kidney disease, gastrointestinal disorders, respiratory disorders, neurological problems, low energy, autonomic dysfunction and dementia.

Treatment of mitochondrial disease is still in its infancy. Aside from symptom-based management, treatment of mitochondrial disease focuses on maintaining optimal health, using preventive measures to mitigate symptom worsening during times of physiologic stress (such as infection, dehydration, or surgery), and avoiding mitochondrial toxins. Most experts use a combination of vitamins, nutritional optimization, and palliative treatments during times of illness and physiologic stress.

Scientific support for the use of vitamin-based and cofactor-based mitochondrial therapies is accumulating. Such pharmacologic supplements are intended to promote critical enzymatic reactions, reduce putative sequelae of excess free radicals, and scavenge toxic acyl coenzyme A (acyl CoA) molecules, which accumulate in mitochondrial disease. Some supplements also may act as alternative energy fuels or may bypass biochemical blocks within the respiratory chain (Parikh et al., Curr Treat Options Neurol. 2009 November; 11(6): 414-430).

However, there exists an ongoing need in the art for a pharmacologic intervention that treats or prevents mitochondrial-related diseases and conditions and enhances mitochondrial efficiency by, e.g., increasing energy production in the form of adenosine triphosphate (ATP), and/or reducing free radical production in an effort to improve, or at least stabilize, disease signs and symptoms. In particular, there is a need in the field for nutritional supplement compositions that effectuate those benefits, and thereby, alleviate, treat or reverse symptoms of mitochondrial disease.

### SUMMARY

The present description relates to nutritional compositions and methods of their use to improve mitochondrial, energy production and efficiency. Surprisingly and unexpectedly, administering a combination comprising an ATP booster and a free radical scavenger (jointly or separately; i.e., successively) improves mitochondrial function more than either alone. As such, nutritional compositions are provided comprising synergistic mixtures that improve health and/or prevent or alleviate at least one symptom of mitochondrial disease.

In one aspect, the nutritional composition comprises a pharmaceutically acceptable carrier, and a synergistically effective amount of a combination of a NAD+ precursor and an ATP booster. In certain embodiments, the synergistically effective amount of the combination of a NAD+ precursor and an ATP booster is comprised or formulated within the same dosage form, which is suitable for administration or consumption by a subject. In other embodiments, the synergistically effective amount of the combination of a NAD+ precursor and an ATP booster is comprised or formulated for co-administration in separate dosage forms, for example, configured for successive or concurrent administration. In certain embodiments, the nutritional composition comprises from about 1% by weight to about 80%% by weight of the total composition of NAD+ precursor in the total composition in combination with from about 1% by weight to about 80% by weight of the total composition of an ATP booster.

In certain embodiments, the NAD+ precursor comprises at least one of nicotinamide riboside, NAD, nicotinic acid, nicotinamide, nicotinic acid mononucleotide, vitamin B3, nicotinamide mononucleotide or a combination thereof.

In certain embodiments, the ATP booster comprises at least one of ancient peat and apple extract, adenosine triphosphate disodium, cordyceps extract, ginseng, extract or fractions of Sphaeranthus indicus, extract or fractions of Coleus aromaticus, extract or fractions of Cissus quadrangular is, extract or fractions of Curcuma longa, extract or fractions of Garcinia mangostana, extract or fractions of Cinnamomum tamala or a combination thereof.

In another aspect, the nutritional composition further comprises an effective amount of an adaptogen.

In certain embodiments, the adaptogen comprises at least one of Rhodiola rosea, Eleutherococcus senticosus, Schisandra chinensis, ginseng, Gynostemma pentaphyllum, ashwagandha or a combination thereof.

In another aspect, the nutritional composition further comprises an effective amount of a transporter of fuel for mitochondrial ATP production.

In certain embodiments, the transporter of fuel for mitochondrial ATP production comprises at least one of acetyl L-Carnitine, Coenzyme Q10 in ubiquinone or ubiquinol forms or a combination thereof.

In another aspect, the nutritional composition further comprises an effective amount an ATP cycle enhancer.

In certain embodiments, the ATP cycle enhancer comprises at least one of R-Alpha-Lipoic acid, carnitine, carnosine, creatine monohydrate, trimethylglycine, policosanol, aspartate, huperzine, medium chain triglycerides, L-arginine-α-ketoglutarate, D-ribose, pyruvate, L-carnitine, L-citrulline, betaine, L-aspartate, glucomannan, succinate, glycine, Camellia sinesis, N-acetyl cysteine, cyanocobalamin, thiamin, pyridoxine, pantothenate, folic acid or a combination thereof.

In another aspect, the nutritional composition further comprises an effective amount of a mitochondria protecting nutrient.

In certain embodiments, the mitochondria protecting nutrient comprises at least one of pterostilbene, resveratrol, astaxanthin, catechin, citicoline, coenzyme Q10, conjugated linoleic acid, cyanidin, diindolylmethane, gamma-linolenic acid, glutathione, glycine, hesperidin, indole-3-carbinol, kaempferol, L-theanine, lycopene, myricetin, naringenin, piceatannol, pterostilbene, quercetin, resveratrol, rutin, S-adenosylmethionine, sulforaphane, superoxide dismutase or a combination thereof.

In a preferred embodiment, the nutritional composition comprises a pharmaceutically acceptable carrier, and NAD+ precursor, ATP booster, adaptogen, transporter of fuel for mitochondrial ATP production, ATP cycle enhancer and mitochondria protecting nutrient. In certain embodiments, the nutritional composition comprises a synergistically effective amount of combination including a NAD+ precursor and an ATP booster, and an effective amount of at least one of an adaptogen, transporter of fuel for mitochondrial ATP production, ATP cycle enhancer, a mitochondrial protecting agent or a combination thereof.

In another aspect, the present application provides a method of enhancing energy in a subject, e.g., a mammal in need thereof, wherein the method comprises administering to the subject a therapeutically effective amount of a nutritional composition as described herein comprising a synergistically effective amount of a combination of a NAD+ precursor and an ATP booster.

In certain embodiments, enhancing energy includes at least one of an increase in mitochondrial energy production (ATP), enhancement of the efficiency of mitochondrial energy production, creation of mitochondrial biogenesis or a combination thereof.

In certain embodiments of the methods as described herein, the mammal is a human.

In another aspect, the present application provides a method of treating a mitochondrial energy disorder or disease in a subject or a mammal in need thereof, wherein the method comprises administering to the subject or a mammal a therapeutically effective quantity of the nutritional compositions of the instant disclosure.

In certain embodiments, the mitochondrial energy disorder or disease comprises Mitochondrial myopathy, Diabetes mellitus and deafness (DAD), Leber's hereditary optic neuropathy (LHON), Leigh syndrome, Neuropathy-ataxia-retinitis pigmentosa-ptosis (NARP), Myoneurogenic gastrointestinal encephalopathy (MNGIE), Myoclonic Epilepsy with Ragged Red Fibers (MERRF), Mitochondrial myopathy-encephalomyopathy-lactic acidosis-stroke-like symptoms (MELAS), Cockayne Syndrome (CS), and mtDNA depletion.

In certain embodiments, the mitochondrial energy disorder or disease comprises Alzheimer's dementia, Parkinson's disease, Huntington disease, Amyotrophic Lateral Sclerosis (ALS), Frontotemporal Dementia (FTD), Mild Cognitive Impairment (MCI), Lewy Body Dementia (LBP), Corticobasal Degeneration (CBD), Macular degeneration, Progressive Supranuclear Palsy (PSP), Vascular Dementia (VaD) and Creutzfeld-Jakob Disease (CJD).

In certain embodiments, the mitochondrial energy disorder or disease comprises Breast cancer, Colorectal cancer, Ovarian cancer, Gastric carcinoma, Hepatocellular cancer, Pancreatic cancer, Prostate cancer, Lung cancer, Renal cell carcinoma, Thyroid cancer, Brain tumors, cervical cancer and esophageal carcinomas and treatments thereof such as chemotherapy.

In certain embodiments, the mitochondrial energy disorder or disease comprises diabetes neuropathy, Acid Lipase Disease, Barth Syndrome (BTHS), Central Pontine Myelinolysis, Farber's Disease, Duchenne muscular dystrophy, Gangliosidoses, Neutral Lipid Storage Disease (NLSD), Mucolipidoses and Mucopolysaccharidoses.

In certain embodiments, the mitochondrial energy disorder or disease includes at least one of sepsis, sarcopenia, arthritis, osteoarthritis or a combination thereof.

In certain embodiments, the mitochondrial energy disorder or disease comprises NASH, non-alcoholic fatty liver disease, alcohol-induced liver disease, viral hepatitis, primary and secondary cholestasis, hemochromatosis, Wilson's disease, Reye's syndrome, Alpers disease, Pearson's marrow pancreas syndrome, Navajo neurohepatopathy, Long chain hydroxyacyl CoA dehydrogenase deficiency, Acute fatty liver of pregnancy, Indian childhood cirrhosis, Idiopathic infantile copper toxicosis, Hereditary hemochromatosis, Neonatal iron storage disease, Tyrosinemia type I and Zellweger syndrome.

In certain embodiments, the present application provides a method of treating a mitochondrial energy disorder or disease in a subject or a mammal, wherein the mammal is human.

In certain embodiments, the present application provides a method of treating a mitochondrial energy disorder or disease in a subject or a mammal, wherein the subject or mammal is receiving chemotherapy treatment.

In another aspect, the present application provides a method of enhancing energy, muscle and/or sport performance in a subject or a mammal in need thereof, wherein the method comprises administering to the subject or a mammal a therapeutically effective quantity of the nutritional compositions as described herein.

In another aspect, the nutritional compositions of the present application further comprise a pharmaceutically-acceptable carrier for said components.

In certain embodiments, a pharmaceutically-acceptable carrier comprises a liquid.

In another aspect, the nutritional compositions of the present application are included in a tablet or a capsule, including a gel capsule, e.g., a liquid-filled gel cap.

In another aspect, the nutritional compositions of the present application are in a dosage form comprising a therapeutically effective amount of a combination as described herein. quantity.

In certain embodiments, the therapeutically effective quantity is from 0.01 mg/kg to 1,000 mg/kg, or from about 0.01 mg/kg to about 75 mg/kg.

In another aspect, the therapeutically effective dosage is included in one or more unitary dosage forms.

In certain embodiments, the unitary dosage form comprises a tablet, a capsule, a soft gel capsule, a liquid, a powder or a combination thereof.

In another preferred embodiment, the nutritional sition comprises in unitary dosage form, e.g., one or more capsules or tablets, about 83.35 mg of Nicotinamide Riboside Chloride, about 250.0 mg of Acetyl L-Carnitine HCL, about 75.0 mg of Ancient peat and Apple extract, about 50.0 mg of R-Alpha Lipoic Acid, about 125.0 mg of Rhodiola rosea 3% rosavins, and about 12.5 mg of pterostilbene. In certain embodiments, the description provides a dietary supplement divided into a dose of 2 capsules per day for a total daily dose of 166.7 mg of Nicotinamide Riboside Chloride, about 500.0 mg of Acetyl L-Carnitine HCL, about 150.0 mg of Ancient peat and Apple extract, about 100.0 mg of R-Alpha Lipoic Acid, about 250.0 mg of Rhodiola rosea 3% rosavins, and about 25 mg of pterostilbene.

In certain embodiments, the amount of nicotinamide riboside in the nutritional compositions of the instant disclosure is from about 0.1 mg to about 1,000 mg.

In certain embodiments, the amount of ATP booster Ancient peat and Apple extract in the nutritional compositions of the instant disclosure is from about 1 mg to about 1,000 mg.

In certain embodiments, the amount of the ATP booster adenosine triphosphate disodium in the nutritional compositions of the instant disclosure is from about 1 mg to about 1,000 mg.

In certain embodiments, the amount of Rhodiola rosea in the nutritional compositions of the instant disclosure is from about 1 mg to about 2,000 mg.

In certain embodiments, the amount of Acetyl L-Carnitine HCL in the nutritional compositions of the instant disclosure is from about 50 mg to about 1,000 mg.

Other embodiments of the instant disclosure will be apparent from the specification and claims.

### DETAILED DESCRIPTION

The present description relates to nutritional compositions and methods of their use to improve mitochondrial energy production and efficiency. Surprisingly and unexpectedly, administering a combination comprising an ATP booster and a free radical scavenger (jointly or separately; i.e., successively) improves mitochondrial function more than either alone. As such, nutritional compositions are provided comprising synergistic mixtures that improve health and/or prevent or alleviate at least one symptom of mitochondrial disease.

Unless otherwise defined, all technical and scientific terms used herein have the same meaning as commonly understood by one of ordinary skill in the art to which this invention belongs. The terminology used in the description is for describing particular embodiments only and is not intended to be limiting of the invention.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The following terms are used to describe the present invention. In instances where a term is not specifically defined herein, that term is given an art-recognized meaning by those of ordinary skill applying that term in context to its use in describing the present invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

The term “about” and the like, as used herein, in association with numeric values or ranges, reflects the fact that there is a certain level of variation that is recognized and tolerated in the art due to practical and/or theoretical limitations. For example, minor variation is tolerated due to inherent variances in the manner in which certain devices operate and/or measurements are taken. In accordance with the above, the phrase “about” is normally used to encompass values within the standard deviation or standard error.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

As used herein, the term “administering” means providing a pharmaceutical agent or composition to a subject, and includes, but is not limited to, administering by a medical professional and self-administering.

The terms “co-administration” and “co-administering” or “combination therapy” can refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are coadministered in combination with at least one additional bioactive agent. In particularly preferred aspects, the co-administration of compounds results in synergistic activity and/or therapy,

The term “effective” can mean, but is in no way limited to, that amount/dose of the active pharmaceutical ingredient, which, when used in the context of its intended use, effectuates or is sufficient to prevent, inhibit the occurrence, ameliorate, delay or treat (alleviate a symptom to some extent, preferably all) the symptoms of a condition, disorder or disease state in a subject in need of such treatment or receiving such treatment. The term effective subsumes all other effective amount or effective concentration terms, e.g., “effective amount/dose,” “pharmaceutically effective amount/dose,” “synergistically effective amount” or “therapeutically effective amount/dose,” which are otherwise described or used in the present application.

The effective amount depends on the type and severity of disease, the composition used, the route of administration, the type of mammal being treated, the physical characteristics of the specific mammal under consideration, concurrent medication, and other factors which those skilled in the medical arts will recognize. The exact amount can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration.

The term “pharmaceutically acceptable” or “pharmacologically acceptable” can mean, but is in no way limited to, entities and compositions that do not produce an adverse, allergic or other untoward reaction when administered to an animal, or a human, as appropriate.

The term “pharmaceutically acceptable carrier” or “pharmacologically acceptable carrier” can mean, but is in no way limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The term “systemic administration” refers to a route of administration that is, e.g., enteral or parenteral, and results in the systemic distribution of an agent leading to systemic absorption or accumulation of drugs in the blood stream followed by distribution throughout the entire body. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect. Administration routes which lead to systemic absorption include, without limitations: intravenous, subcutaneous, intraperitoneal, inhalation, oral, intrapulmonary and intramuscular. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful.

The term “local administration” refers to a route of administration in which the agent is delivered to a site that is apposite or proximal, e.g., within about 10 cm, to the site of the lesion or disease.

The term “subject” is used throughout the specification to describe a cell, tissue, or animal, preferably a human or a domesticated animal, to whom treatment, including prophylactic treatment, with the compositions according to the present invention is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present invention, the term patient refers to a human patient unless otherwise stated or implied from the context of the use of the term.

As used herein, the term “enhancement” of, or to “enhance,” natural energy means to intentionally change the level of one or more energy biomarkers away from either the normal value, or the value before enhancement, in order to achieve a beneficial or desired effect. For example, in a situation where significant energy demands are placed on a subject, it may be desirable to increase the level of ATP in that subject to a level above the normal level of ATP in that subject. Enhancement can also he of beneficial effect in a subject suffering from a disease or pathology such as a mitochondrial disease, in that normalizing an energy biomarker may not achieve the optimum outcome for the subject; in such cases, enhancement of one or more energy biomarkers can be beneficial, for example, higher-than-normal levels of ATP, or lower-than-normal levels of lactic acid (lactate) can be beneficial to such a subject.

The term “ATP booster” as used herein, refers to a substance that enhances energy, as defined above. Examples include ancient peat and apple extract, adenosine triphosphate disodium, cordyceps extract, ginseng, extract or fractions of Sphaeranthus indicus, extract or fractions of Coleus aromaticus, extract or fractions of Cissus quadrangular is, extract or fractions of Curcuma longa, extract or fractions of Garcinia mangostana, extract or fractions of Cinnamomum tamala.

The term “mitochondrial energy disorder or disease” as discussed herein, refers to any disease or condition that is caused by or contributed to by mitochondrial abnormal function. Examples include: Mitochondrial myopathy, Diabetes mellitus and deafness (DAD), Leber's hereditary optic neuropathy (LHON), Leigh syndrome, Neuropathy-ataxia-retinitis pigmentosa-ptosis (NARP), Myoneurogenic gastrointestinal encephalopathy (MNGIE), Myoclonic Epilepsy with Ragged Red Fibers (MERRF), Mitochondrial myopathy-encephalomyopathy-lactic acidosis-stroke-like symptoms (MELAS), Cockayne Syndrome (CS), mtDNA depletion, Alzheimer's dementia, Parkinson's disease, Huntington disease, Amyotrophic Lateral Sclerosis (ALS), Frontotemporal Dementia (FTD), Mild Cognitive Impairment (MCI), Lewy Body Dementia (LBD), Corticobasal Degeneration (CBD), Macular degeneration, Progressive Supranuclear Palsy (PSP), Vascular Dementia (VaD), Creutzfeld-Jakob Disease (CJD), Breast cancer, Colorectal cancer, Ovarian cancer, Gastric carcinoma, Hepatocellular cancer, Pancreatic cancer, Prostate cancer, Lung cancer, Renal cell carcinoma, Thyroid cancer, Brain tumors, cervical cancer, esophageal carcinomas, diabetes neuropathy, Acid Lipase Disease, Barth Syndrome (BTHS), Central Pontine Myelinolysis, Farber's Disease, Duchenne muscular dystrophy, Gangliosidoses, Neutral Lipid Storage Disease (NLSD), Mucolipidoses, Mucopolysaccharidoses, sepsis, sarcopenia, arthritis, osteoarthritis, NASH, non-alcoholic fatty liver disease, alcohol-induced liver disease, viral hepatitis, primary and secondary cholestasis, hemochromatosis, Wilson's disease, Reye's syndrome, Alpers disease, Pearson's marrow pancreas syndrome, Navajo neurohepatopathy, Long chain hydroxyacyl CoA dehydrogenase deficiency, Acute fatty liver of pregnancy, Indian childhood cirrhosis, Idiopathic infantile copper toxicosis, Hereditary hemochromatosis, Neonatal iron storage disease, Tyrosinemia type I, Zellweger syndrome.

The term “NAD+ precursor” as used herein, refers to molecules that can be synthesized into NAD+, either in a de novo pathway from amino acids or in salvage pathways by recycling preformed components such as nicotinamide back to NAD⁺. Examples of NAD+ precursors include nicotinamide riboside, NAD, nicotinic acid, nicotinamide, nicotinic acid mononucleotide, vitamin B3 and nicotinamide mononucleotide.

The term “adaptogen” as used herein, refers to substances, compounds, herbs or practices whereby administration results in stabilization of physiological processes and promotion of homeostasis, an example being by decreased cellular sensitivity to stress. Examples comprise Rhodiola rosea, eleutherococcus senticosus, Schisandra chinensis, ginseng, Gynostemma pentaphyllum, and ashwagandha.

### Compositions

In one aspect, the nutritional composition comprises a pharmaceutically acceptable carrier, and a synergistically effective amount of a combination of a NAD+ precursor and an ATP booster. In certain embodiments, the synergistically effective amount of the combination of a NAD+ precursor and an ATP booster is comprised or formulated within the same dosage form, which is suitable for administration or consumption by a subject. In other embodiments, the synergistically effective amount of the combination of a NAD+ precursor and an ATP booster is comprised or formulated for co-administration in separate dosage forms, for example, configured for successive or concurrent administration. In certain embodiments, the nutritional composition comprises from about 1% by weight to about 80% by weight of the total composition of NAD+ precursor in the total composition in combination with from about 1% by weight to about 80% by weight of the total composition of an ATP booster.

In certain embodiments, the NAD+ precursor comprises nicotinamide riboside. Nicotinamide riboside is a precursor of nicotinamide adenine dinucleotide (NAD), and is a source of Vitamin B3. It has properties that are insulin sensitizing, enhancing to exercise, resisting to negative effects of high-fat diet, and neuroprotecting (Chi and Sauve, Curr Opin Clin Nutr Metab Care. 2013 November; 16(6):657-61.). Nicotinamide riboside is available commercially. In a preferred embodiment of the instant application the source of Nicotinamide riboside is the product. Niagen®, sold by Chromadex, Inc. In general, the dosage of nicotinamide riboside in the compositions of the instant application can be from about 0.1 mg to about 1,000 mg.

In certain embodiments, the ATP booster is ancient peat and apple extract, which is commercially available as elevATP™, purchased from Futureceuticals, Inc. elevATP™ is a combination of a water extract of “ancient peat” (fossilized plants) and apple extract that works with the body natural energy-producing mechanisms to stimulate production of ATP. Recent human clinical research indicates elevATP stimulates production of ATP at the mitochondrial level, without increasing lactic acid or reactive oxygen species levels (Reyes-Izquierdo et al., Journal of Aging Research & Clinical Practice, 01/2014; 1(1):1-5.). In general, the dosage of elevATP™ in the compositions of the instant application can be from about 1 mg to about 1,000 mg.

In certain embodiments, the ATP booster is adenosine triphosphate disodium, which is commercially available as PeakATP®, purchased from TSI Health Sciences, Inc. PeakATP® is stabilized orally available ATP disodium, which can directly increase the levels of extracellular ATP in humans and has been clinically shown to increase certain measures of strength and muscular energy (Wilson J M, et al. Effects of oral adenosine-5′-triphosphate supplementation on athletic performance, skeletal muscle hypertrophy and recovery in resistance-trained men. Nutrition and Metabolism. 2013, 10:57. Rathmacher J A, et al. Adenosine-5′-triphosphate (ATP) supplementation improves low peak muscle torque and torque fatigue during repeated high intensity exercise sets. Journal of the International Society of Sports Nutrition. 2012, 9:48; and U.S. Pat. Nos. 5,227,371; 5,049,372; 8,841,350; 8,106,184; 8,114,626; 8,197,807; 8,383,086; and 7,776,326 all of which are incorporated herein by reference in their entirety.

In certain embodiments, the amount of the ATP booster adenosine triphosphate disodium is from about 1 mg to about 1,000 mg. In general, the dosage of PeakATP® in the compositions of the instant application can be from about 1 mg to about 1,000 mg.

In another aspect, the nutritional composition further comprises an effective amount of an adaptogen. In a certain embodiments, the adaptogen is Rhodiola rosea. Rhodiola rosea (commonly golden root, rose root, roseroot, western roseroot, Aaron's rod, arctic root, king's crown, Lignum rhodium, orpin rose) is a perennial flowering plant in the family Crassulaceae.

R. rosea may be effective for improving mood and alleviating depression. Some evidence suggests that the herb may be helpful for enhancing physical performance and alleviating mental fatigue. Rosavin, rosarin, rosin and salidroside are among suspected active ingredients of R. rosea. In a preferred embodiment, the nutritional composition of the instant application comprises Rhodiola rosea 3% rosavins. Rhodiola rosea is available commercially. In general, the dosage of Rhodiola rosea in the compositions of the instant application can be from about 1 mg to about 2,000 mg daily.

In another aspect, the nutritional composition further comprises an effective amount of transporter of fuel for mitochondrial ATP production. In an exemplary embodiment, the transporter of fuel for mitochondrial ATP production is Acetyl L-Carnitine. Acetyl-L-carnitine or ALCAR, is an acetylated form of L-carnitine. It is naturally produced by the body, although it is often taken as a dietary supplement. Acetylcarnitine is broken down in the blood by plasma esterases to carnitine which is used by the body to transport fatty acids into the mitochondria for breakdown. ALCAR is an acetylated derivative of L-carnitine. During strenuous exercise, a large portion of L-carnitine and unused acetyl-CoA are converted to ALCAR and CoA inside mitochondria by carnitine O-acetyltransferase. The ALCAR is transported outside the mitochondria where it converts back to the two constituents.

L-carnitine is cycled back into the mitochondria with acyl groups to facilitate fatty acid utilization, but excess acetyl-CoA may block it. Excess acetyl-CoA causes more carbohydrates to be used for energy at the expense of fatty acids. This occurs by different mechanisms inside and outside the mitochondria. ALCAR transport decreases acetyl-CoA inside the mitochondria, but increases it outside. Glucose metabolism in diabetics improves with administration of either ALCAR or L-carnitine. ALCAR decreases glucose consumption in favor of fat oxidation in non-diabetics. A portion of L-carnitine is converted to ALCAR after ingestion in humans. Acetyl L-Carnitine is available commercially. In general, the dosage of Acetyl L-Carnitine in the compositions of the instant application can be from about 50 mg to about 1,000 mg daily.

In another aspect, the nutritional composition further comprises an effective amount of ATP cycle enhancer. In certain embodiments, the ATP cycle enhancer is R-Alpha-Lipoic acid. Lipoic acid (LA), also known as α-lipoic acid and alpha lipoic acid (ALA) and thioctic acid is an organosulfur compound derived from octanoic acid. ALA is made in animals normally, and is essential for aerobic metabolism. Lipoic acid is cofactor for at least five enzyme systems. Two of these are in the citric acid cycle through which many organisms turn nutrients into energy. R-Alpha-Lipoic acid is available commercially.

In yet another aspect, the nutritional composition further comprises an effective amount of mitochondria protecting nutrient. In certain embodiments, the mitochondria protecting nutrient comprises pterostilbene. Pterostilbene (trans-3,5-dimethoxy-4-hydroxystilbene) is a natural dietary compound and the primary antioxidant component of blueberries. It has increased bioavailability in comparison to other stilbene compounds, which may enhance its dietary benefit and possibly contribute to a valuable clinical effect. Multiple studies have demonstrated the antioxidant activity of pterostilbene in both in vitro and in vivo models illustrating both preventative and therapeutic benefits. The antioxidant activity of pterostilbene has been implicated in anticarcinogenesis, modulation of neurological disease, anti-inflammation, attenuation of vascular disease, and amelioration of diabetes (McCormack and McFadden, Oxid Med Cell Longev. 2013; 2013: 575482). Pterostilbene is available commercially. In a preferred embodiment, the pterostilbene is PteroPure® made by ChromaDex.

In certain embodiments, therapeutic pharmaceutical compositions are provided that comprise therapeutically effective amount of a composition comprising a synergistically effective amount of a combination (i) at least one NAD+ precursor and (ii) an ATP booster, formulated together with one or more pharmaceutically acceptable carriers. In certain embodiments, the pharmaceutically acceptable carrier comprises at least one of, e.g., a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of the present application can be administered to humans and other animals orally. Thus, in a preferred embodiment, the present application provides pharmaceutical formulations for oral administration.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid compositions of a similar type may also be employed as fillers in soft and hard-filled gelatin capsules using such excipients as lactose or milk sugar as well as high molecular weight polyethylene glycols and the like.

The active compounds can also he in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such as magnesium stearate and microcrystalline cellulose. In certain embodiments, the dosage form is a two-piece hardshell gelatin capsule; a two-piece hardshell vegetable gelatin capsule; a soft gelatin capsule; a vegetarian soft gelatin capsule; or a tablet. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

### Methods and Administration

In one aspect, the present application provides a method of enhancing natural energy in a subject or a mammal in need thereof. The method comprises administering to the subject or a mammal a composition comprising an effective amount of at nutritional composition as described herein, wherein the composition comprises a synergistically effective amount of at least one NAD+ precursor and at least one ATP booster.

In certain embodiments, the enhancement of natural energy comprises increase in mitochondrial energy production (ATP), enhancement of the efficiency of mitochondrial energy production and creation of mitochondrial biogenesis.

In another aspect, the present application provides a method of treating a mitochondrial energy disorder or disease in a subject or a mammal in need thereof. The method includes the step of administering to the subject or a mammal a composition comprising an effective synergistic amount of at least one NAD+ precursoe and at least one ATP booster.

In another aspect, the present application provides a method of enhancing energy, muscle performance and/or sport performance in a subject or a mammal in need thereof. The method includes the step of administering to the subject or a mammal a composition comprising an effective synergistic amount of at least one NAD+ precursor and at least one ATP booster.

In a preferred embodiment, the NAD+ precursor is nicotinamide riboside. In certain embodiments, the amount of nicotinamide riboside can be from about 0.1 to about 1,000 mg.

In a preferred embodiment, the ATP booster is ancient peat and apple extract. In certain embodiments, the amount of Ancient peat and apple extract can be from about 1 mg to about 1,000 mg.

In a preferred embodiment, the nutritional composition is administered on a daily basis.

In certain embodiments, the pharmaceutical composition or formulation is formulated for oral administration. In certain embodiments, the pharmaceutical formulation is in the form of a tablet. In certain embodiments, the pharmaceutical formulation is in the form of a two-piece capsule. In certain embodiments, the pharmaceutical formulation is in the form of dosage unit distributions such as tablets, coated tablets, hard or soft gelatin capsules.

In certain embodiments, the pharmaceutical formulation is in a liquid dosage form.

### EXAMPLE 1

An example of a composition of the present application is a dietary supplement containing the following:

A capsule is filed with:
a. 83.3500 mg of Nicotinamide Riboside Chloride (Niagen®)
b. 250.000 mg of Acetyl L-Carnitine HCL
c. 75.000 mg of Ancient peat and Apple extract (elevATP®)
d. 50.000 mg of R-Alpha Lipoic Acid
e. 125.000 mg of Rhodiola rosea 3% rosavins
f. 12.500 mg of pterostilbene (PteroPure®)
g. With a total of 595.850 mg per capsule of active ingredients.
h. Other ingredients: Hypromellose (vegetarian capsules), microcrystalline cellulose, vegetable source magnesium stearate.
i. Suggested use: 2 capsules daily with a large glass of water.

1. A nutritional composition comprising a pharmaceutically acceptable carrier, and a synergistically effective amount of a combination of NAD+ precursor and an ATP booster.

2. The composition of claim 1, wherein the NAD+ precursor comprises at least one of nicotinamide riboside, NAD, nicotinic acid, nicotinamide, nicotinic acid mononucleotide, vitamin B3, nicotinamide mononucleotide or a combination thereof.

3. The composition of claim 1, wherein the ATP booster comprises at least one of ancient peat and apple extract, adenosine triphosphate disodium, cordyceps extract, ginseng, extract or fractions of Sphaeranthus indicus, extract or fractions of Coleus aromaticus, extract or fractions of Cissus quadrangular is, extract or fractions of Curcuma longa, extract or fractions of Garcinia mangostana, extract or fractions of Cinnamomum tamala or a combination thereof.

4. The composition of claim 1, further comprising an effective amount of an adaptogen.

5. The composition of claim 4, wherein the adaptogen comprises at least one of Rhodiola rosea, Eleutherococcus senticosus, Schisandra chinensis, ginseng, Gynostemma pentaphyllum, ashwagandha or a combination thereof.

6. The compositions of claim 1, further comprising an effective amount of a transporter of fuel for mitochondrial ATP production.

7. The composition of claim 6, wherein the transporter of fuel for mitochondrial ATP production comprises at least one of acetyl L-Carnitine, Coenzyme Q10 in ubiquinone or ubiquinol forms or a combination thereof.

8. The compositions of claim 1, further comprising an effective amount of an ATP cycle enhancer.

9. The composition of claim 8, wherein the ATP cycle enhancer comprises at least one of R-Alpha-Lipoic acid, carnitine, carnosine, creatine monohydrate, trimethylglycine, policosanol, aspartate, huperzine, medium chain triglycerides, L-arginine-α-ketoglutarate , D-ribose, pyruvate, L-carnitine, L-citrulline, betaine, L-aspartate, glucomannan, succinate, glycine, Camellia sinesis, N-acetyl cysteine, cyanocobalamin, thiamin, pyridoxine, pantothenate, folic acid or a combination thereof.

10. The compositions of claim 1, further comprising an effective amount of a mitochondria protecting nutrient.

11. The composition of claim 10, wherein the mitochondria protecting nutrient comprises at least one of pterostilbene, resveratrol, astaxanthin, catechin, citicoline, coenzyme Q10, conjugated linoleic acid, cyanidin, diindolylmethane, gamma-linolenic acid, glutathione, glycine, hesperidin, indole-3-carbinol, kaempferol, L-theanine, lycopene, myricetin, naringenin, piceatannol, pterostilbene, quercetin, resveratrol, rutin, S-adenosylmethionine, sulforaphane, superoxide dismutase or a combination thereof.

12. A method of enhancing energy in a subject or a mammal in need thereof, wherein the method comprises administering to the subject or a mammal a therapeutically effective quantity of the composition according to claim 1.

13. The method of claim 12, wherein the enhancement of energy comprises increase in mitochondrial energy production (ATP), enhancement of the efficiency of mitochondrial energy production and creation of mitochondrial biogenesis.

14. The method of claim 12, wherein the mammal is human.

15. A method of treating a mitochondrial energy disorder or disease in a subject or a mammal in need thereof, wherein the method comprises administering to the subject or a mammal a therapeutically effective quantity of the composition according to claim 1.

16. The method of claim 15, wherein the mitochondrial energy disorder or disease comprises Mitochondrial myopathy, Diabetes mellitus and deafness (DAD), Leber's hereditary optic neuropathy (LHON), Leigh syndrome, Neuropathy-ataxia-retinitis pigmentosa-ptosis (NARP), Myoneurogenic gastrointestinal encephalopathy (MNGIE), Myoclonic Epilepsy with Ragged Red Fibers (MERRF), Mitochondrial myopathy-encephalomyopathy-lactic acidosis-stroke-like symptoms (MELAS), Cockayne Syndrome (CS) and mtDNA depletion.

17. The method of claim 15, wherein the mitochondrial energy disorder or disease comprises Alzheimer's dementia, Parkinson's disease, Huntington disease, Amyotrophic Lateral Sclerosis (ALS), Frontotemporal Dementia (FTD), Mild Cognitive Impairment (MCI), Lewy Body Dementia (LBD), Corticobasal Degeneration (CBD), Macular degeneration, Progressive Supranuclear Palsy (PSP), Vascular Dementia (VaD), and Creutzfeld-Jakob Disease (CJD).

18. The method of claim 15, wherein the mitochondrial energy disorder or disease comprises Breast cancer, Colorectal cancer, Ovarian cancer, Gastric carcinoma, Hepatocellular cancer, Pancreatic cancer, Prostate cancer, Lung cancer, Renal cell carcinoma, Thyroid cancer, Brain tumors, cervical cancer and esophageal carcinomas and treatments related to cancer such as chemotherapy.

19. The method of claim 15, wherein the mitochondrial energy disorder or disease comprises diabetes neuropathy, Acid Lipase Disease, Barth Syndrome (BTHS), Central Pontine Myelinolysis, Farber's Disease, Duchenne muscular dystrophy, Gangliosidoses, Neutral Lipid Storage Disease (NLSD), Mucolipidoses and Mucopolysaccharidoses.

20. The method of claim 15, wherein the mitochondrial energy disorder or disease comprises sepsis.

21. The method of claim 15, wherein the mitochondrial energy disorder or disease comprises sarcopenia, arthritis and osteoarthritis.

22. The method of claim 15, wherein the mitochondrial energy disorder or disease comprises NASH, non-alcoholic fatty liver disease, alcohol-induced liver disease, viral hepatitis, primary and secondary cholestasis, hemochromatosis, Wilson's disease, Reye's syndrome, Alpers disease, Pearson's marrow pancreas syndrome, Navajo neurohepatopathy, Long chain hydroxyacyl CoA dehydrogenase deficiency, Acute fatty liver of pregnancy, Indian childhood cirrhosis, Idiopathic infantile copper toxicosis, Hereditary hemochromatosis, Neonatal iron storage disease, Tyrosinemia type I and Zellweger syndrome.

23. A method of enhancing energy and muscle and/or sports performance in a subject or a mammal in need thereof, wherein the method comprises administering to the subject or a mammal a therapeutically effective quantity of the composition according to claim 1.

24. The method of claim 23, wherein the method enhances sport performance.

25. The compositions of claim 1, further comprising a pharmaceutically-acceptable carrier for said components.

26. The compositions of claim 1, wherein said carrier comprises a liquid.

27. The compositions of claim 1, wherein the composition is included in a tablet or a capsule.

28. A dosage form comprising a therapeutically effective quantity of the composition of claim 1.

29. The dosage form of claim 28, wherein the therapeutically effective quantity is from 0.01 mg/kg to 75 mg/kg.

30. The dosage form of claim 29, wherein the dosage is included in one or more unitary dosage forms.

31. The dosage form of claim 30, wherein the unitary dosage form comprises a tablet, a capsule, a soft gel capsule, a liquid, a powder or a combination thereof.

32. The composition of claim 10 according to the capsule dosage form of claim 33 comprising about 83.35 mg of Nicotinamide Riboside Chloride, about 250.0 mg of Acetyl L-Carnitine HCL, about 75.0 mg of Ancient peat and Apple extract, about 50.0 mg of R-Alpha Lipoic Acid, about 125.0 mg of Rhodiola rosea 3% rosavins, and about 12.5 mg of pterostilbene.

33. The composition of claim 1, wherein the composition comprises from about 0.1 mg to about 1,000 mg of nicotinamide riboside.

34. The compositions of claim 1, wherein the composition comprises from about 0.1 mg to about 2,000 mg of Rhodiola rosea.

35. The compositions of claim 1, wherein the composition comprises from about 1 mg to about 1,000 mg ancient peat and apple extract.

36. The compositions of claim 1, wherein the composition comprises from about 1 mg to about 1,000 mg of adenosine triphosphate disodium.

37. The compositions of claim 1, wherein the composition comprises from about 50 mg to about 2,000 mg of Acetyl L-Carnitine.
